(19) United States
(12) Patent Application Publication
(10) Pub. No.: US 2002/0022057 A1
(43) Pub. Date: Feb. 21, 2002

(54) Oral delivery of pharmaceuticals via encapsulation

(76) Inventors: Alyce S. Battey, West Springfield, MA (US); Jacob Battey, West Springfield, MA (US)

Correspondence Address: Parker & DeStefano, Suite 300, 300 Preston Avenue, Charlottesville, VA, 22902, US

(21) Appl. No.: 09/931,793

(22) Filed: Aug. 17, 2001

Related U.S. Application Data
(60) Provisional application No. 60/225,877, filed on Aug. 17, 2000.

(51) Int. Cl. A61K 9/14; A61K 9/16

(52) U.S. Cl. 424/490; 514/282; 514/270; 514/210.09

(57) ABSTRACT

An alternate drug delivery system for dissolution of pharmaceuticals in the mouth wherein a therapeutically effective amount of a drug is encapsulated using an encapsulation method. Encapsulation reduces the perceived off flavors of drugs, allowing the active components to dissolve pleasantly in the mouth. This allows more rapid absorption of the active compounds through the oral cavity compared to traditional tablets, which require breakdown and absorption in the gastrointestinal tract. The delivery system can be incorporated into a variety of applications, such as breath mint tablets or chewing gum. Benefits of this invention include portability and the ability to take pharmaceuticals without water and without the off taste of chewable tablets, thereby leading to increased patient compliance.

### BACKGROUND OF THE INVENTION

1. Field of the Invention

This invention relates to a new delivery system for pharmaceuticals using encapsulation technology and a process for its preparation. In particular, the invention relates to a process for preparing products that are capable of dissolving in the mouth. This leads to more rapid absorption of the pharmaceutical components than traditional delivery methods. The compositions are designed to mask the taste associated with many drugs and provide a pleasant flavor sensation.

### 2. BRIEF DESCRIPTION OF THE PRIOR ART

The traditional delivery method for pharmaceutically-active compounds is a tablet or capsule which is designed to be swallowed whole and absorbed in the gastrointestinal (GI) tract.

Many drugs, including all non-steroidal anti-inflammatory drugs such as aspirin, can cause gastrointestinal upset, heartburn, indigestion, ulcers, and gastric bleeding. [Croft et al., Br. Med. J. 1:137 (1967); Petty et al., Ann. Intern. Med. 130:14-22 (1999)]. Compounds can be buffered to minimize the contact time with the stomach lining.

Many products are coated to prevent absorption in the stomach and to minimize the unacceptable taste characteristics associated with many drugs. Enteric coatings are often employed, which allow the analgesic (e.g. aspirin) to pass through the stomach and dissolve in the small intestine. The more alkaline environment of the intestines dissolves the enteric coating allowing the aspirin to be released. The result is slow and/or incomplete absorption, leading to delayed therapeutic effects.

There are many examples of enteric coatings, as summarized in U.S. Pat. No. 4,755,387. U.S. Pat. No. 3,524,910, discloses coating analgesics with ethyl cellulose in a weight amount relative to the amount of analgesic of from 1:22 to 1:50. U.S. Pat. No. 3,656,997 discloses analgesic containing gelatin capsules having a first coating of an enteric material. U.S. Pat. No. 3,691,090 discloses a method of encapsulating aspirin cores with an enteric polymer. U.S. Pat. No. 3,906,086 discloses a particle having an aspirin core and an enteric phthalate coating. U.S. Pat. No. 4,308,251 discloses aspirin tablets containing aspirin, an enteric material and an erosion promoting agent such as corn starch.

Additional references that disclose analgesic coatings include U.S. Pat. No. 2,953,497 which discloses analgesic granules having a cellulose acetate phthalate coating; U.S. Pat. No. 3,166,476 which discloses aspirin particles which have a first coating of a material such as gelatin and a second enteric coating; and British Pat. No. 1,129,811 which discloses aspirin particles which have a coating of a polysalicylide and possibly a second, unspecified coating.

The use of lipids to provide a sustained release of therapeutic agents were disclosed by U.S. Pat. No. 2,921,883 using a mixture of a lipid (e.g., glyceryl tri-dihydroxystearate) and a cellulose derivative; U.S. Pat. No. 3,147,187 using drug mixed with a fat (e.g., glyceryl tristearate), and a swellable gum or proteinaceous material; U.S. Pat. No. 3,402,240 using lipid matrix of carnauba wax, candelilla wax, esparto wax, or ouricury wax; U.S. Pat. No. 4,132,753 using a wax-like material in conjunction with radiation heating; U.S. Pat. No. 4,375,468 using waxes, fats, cellulose esters alone or in various combinations; U.S. Pat. No. 4,483,847 using a mixture of high and low melting point lipids; U.S. Pat. Nos. 4,755,387; 5,891,476 using a wax and emulsifier; and U.S. Pat. No. 5,728,403 using an unique combination of triglycerides that melt at body temperatures and a polymer that dissolves in the acidic environment of the stomach.

Alternative delivery systems for those patients who have difficulty swallowing tablets include chewable tablets and liquid solutions. These forms are particularly beneficial to certain classes of ill people, children, elderly people, and even veterinary patients.

Chewable dosage forms for drug delivery are well known to the pharmaceutical industry. Conventional chewable tablets consist of the medicinal agent and various ingredients, such as sugars, flavors, and colors. A common problem associated with chewable tablets is the unpleasant taste of the active components. These include metallic tastes, acidity, bitterness, burning in the back of the throat, and unpleasant odors. A great deal of research has been conducted to improve the taste of chewable tablets.

Some of the research has focused around coating the unpleasant flavors. U.S. Pat. No. 4,786,502 discloses the use of lipid material to mask bad tasting drugs in chewable form. U.S. Pat. Nos. 4,609,543 and 5,320,848 disclose the use of lipid materials and special processes to provide intimate contact of the lipid with the medicament to achieve superior taste masking. U.S. Pat. No. 4,882,152 discloses a coating using glycerides, lecithin, polyoxylalkylenes, or polyalkylene glycols gelatin, sweetener, glycerin, and water. U.S. Pat. No. 4,786,508 discloses using a polymeric coating containing copolymer with residues of (meth)acrylic esters and dimethy; aminoethyl (meth)acrylate. U.S. Pat. No. 4,894,233 discloses coating the medicament particles with a hydrophobic matrix to mask the taste of the drug. U.S. Pat. No. 4,975,270 uses elastomer-encased active ingredients dispersed in an intensely flavored chewing gum base to mask the taste of medicaments. U.S. Pat. No. 5,380,535 discloses a drug intimately dispersed or dissolved in a pharmaceutically-acceptable lipid that is soluble at room temperatures. The utilization of lipids to mask the taste of the drugs often imparts a greasy taste and dispersants, such as sugar and emulsifiers, are used to minimize this sensation. U.S. Pat. No. 5,753,255 discloses the use of tricaprin (capric triglyceride) which requires no dispersant material to produce a superior tasting tablet. U.S. Pat. No. 4,851,226 discloses coating active ingredients with a blend of cellulose acetate or cellulose actetate butyrate and polyvinylpyrrolidone. U.S. Pat. No. 5,494,681 discloses the use of a wax and hydrophobic polymer to deliver a tasteless pharmaceutical.

Another way to improve the taste of chewable tablets is to process the medicament into rotogranules (U.S. Pat. No. 5,215,755) and to coat these granules with a mixture of hydroxyethyl cellulose and hydroxypropyl methylcellulose (U.S. Pat. No. 5,460,825). This disperses the medicament into smaller pieces throughout the product and reduces the perceived strength of the bitter components.

Off flavors associated with drugs can often be overpowered with strong flavors and sweeteners, as disclosed in U.S. Pat. No. 5,013,716. Taste-masking additives have also been found to minimize the perception of unpleasant components. U.S. Pat. No. 4,758,424 discloses masking a bitter tasting decongestant with a complex aluminum silicate comprised of silicon dioxide, magnesium oxide, and aluminum oxide. U.S. Pat. No. 4,632,821 discloses the use of a complex magnesium trisilicate to reduce the bitterness of decongestants. U.S. Pat. Nos. 4,971,791 and 5,286,489 disclose using a copolymer with a plurality of carboxylic acid and ester groups to create a drug-polymer matrix with an amine or amido containing active compound. U.S. Pat. Nos. 4,910,023 and 5,681,577 disclose the use of silicon dioxide to mask poor tasting drugs. U.S. Pat. No. 5,599,556 discloses the use of a prolamine coating to mask the unpleasant tastes associated with orally administered drugs.

Chewable forms of tablets are known to have a rapid onset of bioactivity compared to conventional tablets that are swallowed (U.S. Pat. Nos. 4,820,523; 6,060,078, Daruwala, 1980). This is because the active components are presented to the GI tract in smaller pieces and often times in a solution with saliva. This leads to more rapid absorption than conventional solid tablets which require disintegration and dissolution.

Liquid forms have an even faster rapid absorption rate than conventional solid delivery systems. Liquid forms include suspensions, syrups, and elixirs. A significant challenge while formulating liquid medicines includes masking the off flavors of the medicament. Furthermore, liquid medications are less portable and are inconvenient to administer.

All of these forms of swallowed medications require absorption through the stomach or intestine. Once the drug is absorbed, it passes through the portal vein, into the liver, and then to the heart and arterial circulation, which distributes the drug throughout the body. The drug is often diluted by gastrointestinal secretions and stomach contents. The drug can also interact with normal constituents of the GI tract, food stuffs, or other drugs, decreasing the absorption. Often times, the efficacy of certain drugs is reduced by the enzymes and environmental conditions of the GI tract. Furthermore, the liver, as the body's major organ of detoxification, can metabolize the drug, minimizing its therapeutic effect.

There are many occasions where the absorption rate is critical to therapeutic treatment. For example, it is widely accepted that antithrombotic agents such as aspirin can be used to treat people who are having a heart attack. Aspirin and other antithrombotic agents have been reported to inhibit cyclooxygenase, an enzyme found to induce platelet aggregation and constriction of blood vessels [Burch et al., J. Clin. Invest. 61:314 (1978); Majerus, J. Clin. Invest. 72:1521 (1983); Roth et al., J. Clin. Invest. 56:624 (1975); FitzGerald et al., J. Clin. Invest. 71:676 (1983); Preston et al., N. Engl. J. Med. 304:76 (1981)]. Soluble aspirin and mouth-dispersible aspirin has been recommended over plain aspirin or enteric-coated aspirin because of their rapid absorption rates [Muir et al., Curr Med Res Opin. 13(10):547-53 (1997)].

Examples of drug delivery systems that are not swallowed include parenteral (by injection), transdermal (through the skin), sublingual (under the tongue), and buccal (between the cheek and gum) administration. Parenteral administration is the most rapid form since the drug is injected directly into the blood stream and does not require any absorption. This is a widely used method for many applications but is not preferred by patients for their daily medication needs, for obvious reasons.

There has been a great deal of research focusing on the transdermal administration of drugs. U.S. Pat. No. 4,654,209 describes creams containing analgesics which when applied to the skin resulted in positive blood and urine tests for the active ingredient. Topical applications of the drugs followed by absorption through the unbroken skin are usually found to be ineffective, in part because the rate of absorption or penetration through the skin is too slow to be effective and because the molecular size of the analgesics is quite large.

Compounds that enhance the rate of penetration have been proposed. U.S. Pat. No. 4,948,588 discloses percutaneous absorption accelerators using analgesics, such as morphine, codeine, and aspirin. Aprotic solvents such as dimethylsulfoxide lead to increased absorption [Scheuplein, J. Investig. Dermatology, Vol. 67, 31-38 (1976)], but produce side effects including inflammation and soreness of the skin, as well as foul breath and body odor.

U.S. Pat. No. 4,476,115 discloses an analgesic composition applied to the skin together with or subsequent to the application of a non-toxic water-soluble sulfite. Percutaneous or transdermal absorption of the analgesic was observed within a few hours of administration, usually within 20-30 minutes.

U.S. Pat. No. 6,071,896 discloses a percutaneous administration of aspirin for suppression of thromboxane levels. Aspirin is applied topically to the skin without the gastric effects normally associated with aspirin therapy. Poor bioavailability was shown by monitoring platelet cycloxygenase activity and plasma drug levels. Aspirin applied to the skin absorbed very slowly, resulting in delayed onset of activity. Furthermore, skin reactions, including erythema and peeling, were noted in 30% of the subjects.

Sublingual and buccal administrations allow the drug to be absorbed in the mucosal lining of the mouth, either under the tongue or in the area between the cheek and gum. The traditional forms of administration are compressed buccal tablets and compressed sublingual tablets.

Lozenges are defined as flavored medicated dosage forms intended to be sucked and held in the mouth or pharynx [Drug Ther. Bull. Lozenges, Mouthwashes, and Gargles, 10:33 (1972)]. Lozenges are traditionally hard candies that provide a slow, uniform release of medicine directly onto the affected mucous membrane (Peters, 1980). The less common form of lozenges is a compressed tablet.

Drugs that can be absorbed in the mouth enter the bloodstream more rapidly and at a higher concentration than traditional swallowed tablets [Ansel, 1981; Conine and Pikal, 1980]. The mucosal lining of the mouth is highly vascular and moves the drug directly into the heart and arterial circulation without first passing through the liver.

Absorption of therapeutic agents through the mouth can be very effective, but is utilized by only a few types of drugs. Lozenges commonly include medicaments that require contact with the mucous membrane, such as decongestants and cough suppressants. It is difficult to include many medicines into traditional hard candy lozenges because of the high temperature used (135 to 150°C.), poor solubility of many drugs, and their high melting points. Furthermore, the moisture content of hard candies (0.5 to 1.5%) and their hygroscopic characteristics cause many medicines to degrade.

Sublingual and buccal applications are even more limited and include nitroglycerin and certain steroid sex hormones. Drugs such as amphetamines, adrenaline, barbiturates, analeptics, and alkaloids such as morphine, cocaine, and codeine, have been explored (Beckett et al., 1968; Walton, 1944; Walton and Lacey, 1935).

The general application of orally absorbing drugs has not been extensively studied. It has been suggested that this is due to the bitter taste imparted by many drugs and the psychological barriers associated with holding drugs in the mouth (Ansel, 1981).

An alternative delivery system is needed that provides specific therapeutic effects without the side effects associated with current delivery forms. It would be extremely desirable to provide consumers with a portable, convenient, pleasant tasting delivery form where the therapeutic effects are faster acting than those currently on the market. Improved patient compliance is expected as a result of these benefits.

### SUMMARY OF THE INVENTION

This invention provides a delivery system which allows the medicament to be absorbed in the oral cavity, leading to a more rapid response to the drug. Specific therapeutic effects, such as the analgesic and antithrombotic properties of aspirin, would be obtained without exposing the GI tract to high local concentrations of the drug. This invention provides a pleasant experience for patients on drug therapy regimens and allows patients with gastric intolerance, or duodenal or gastric ulcers to benefit from many medicines. Furthermore, this invention provides customers with a portable delivery form which is easy to administer and pleasant tasting.

In accordance with the present invention, an alternative drug delivery system is proposed that is capable of absorption in the oral cavity and masking the taste of the drug through encapsulation.

The product can be administered orally and allowed to remain in the mouth, instead of swallowing or chewing the tablet. This allows the active compounds to absorb through the lining in the oral cavity, leading to more rapid absorption than traditional delivery systems.

The encapsulated components can be used in a variety of applications, including but not limited to, compressed tablets and chewing gum.

### DESCRIPTION OF THE PREFERRED EMBODIMENTS OF THE INVENTION

Many kinds of therapeutic agents can be utilized in the present invention, such as anesthetics, decongestants, antihistamines, antitussives, antibotics, analgesics, alkaloids (morphine, codeine, caffeine, cocaine), amphetamines, hormones (adrenaline, epinephrine), barbiturates, and analeptics. Medicines with a high oil/water partition coefficient or good oil solubility are effective to administer through oral absorption. This is because absorption through the oral mucosa is a passive diffusion of the un-ionized form of the drug from the aqueous phase (in the saliva) to the lipid phase (in the membrane) [Beckett et al., 1968].

The level of medicament is dependent on several factors, including the desired dosage, method of encapsulation, absorption characteristics, and desired tablet size. Compressed tablets are rarely greater than 25 to 30% of the final tablet weight, because of the flavor, mouth-feel, and other organoleptic issues (Peters, 1980). The benefit of the present invention is that higher levels of the drug can be used, because the encapsulation helps to mask the off flavors. An additional benefit of the present invention is that lower levels of drugs are required to achieve the same therapeutic effect because of the greater absorption rates.

The medicament(s) can be incorporated into the basic system of this invention along with flavor(s) and other optional ingredients. These include natural and artificial sweeteners, colors, preservatives, fillers, binders, lubricants, suspending agents, acids, buffers, stabilizing agents, and any other ingredients familiar to those skilled in the art of pharmaceutical and confectionery formulation.

The use of flavors to mask the off taste associated with many medicines is widespread. It has been recommended that flavors be selected that offset the tastes associated with certain drugs Daruwala, 1980). For example, acidic or sour drugs, such as aspirin, are best masked with acidic flavors, such citrus, berry flavors, licorice, and root beer. Bitter drugs are best masked by mint, licorice, cherry, nut, and chocolate flavors. Metallic flavors can be masked well with grape.

The use of sweeteners to mask the unpleasant taste of drugs is also widespread (Daruwala, 1980). Sweeteners such as sucrose, fructose, dextrose, lactose, mannitol, sorbitol, maltose, xylitol, saccharin and saccharin sodium, cyclamates, neotame, aspartame, sucralose, acesulfame potassium can be utilized. Artificial sweeteners often have an extremely high degree of sweetness, which comes in handy when formulating pleasant tasting products. Since only small quantities of the high intensity sweetener are required to mask very bitter components, a prohibitively large tablet is not required.

Certain sweeteners can impart their own characteristics to the product. For example, cooling sensation and smooth mouth feel are associated with tablets containing sorbitol and dextrose while those containing artificial sweeteners, such as aspartame and saccharin, can impart bitterness. A variety of masking agents are available to mask undesirable flavors. Even low levels of sodium chloride can be used to increase the sweetness perception and overcome off flavors.

The use of flavoring agents and sweeteners to mask the off taste has been criticized when making sublingual or buccal tablets (Conine and Pikal). This is because the compounds increase the flow of saliva. Certain drugs are designed not to be swallowed since the absorption rate through the GI tract can be significantly reduced or the active components can be destroyed by the liver. This must be taken into account during the selection of the medicament, flavors and sweeteners, and the encapsulation system. This invention best serves as an alternate delivery system for those types of drugs that are currently swallowed.

The process of encapsulating therapeutic substances can be achieved using many different types of technology, since many known encapsulation processes can create a palatable product that can be absorbed in the mouth. The critical component is to encapsulate the drug and flavors into a stable form that protects the active component from chemical interactions and reactions caused by environmental abuses, such as temperature fluctuations, moisture, and light.

Microencapsulation technologies currently employed by the food, flavor, and pharmaceutical industries are summarized by Dziezak (1988), Heath (1978), and Tan (1995). These include spray drying, air suspension coating, extrusion, spray cooling and chilling, emulsion and microemulsion, adsorption, centrifugal extrusion, rotational suspension separation, rotogranulation, coacervation, continuous crystallization or cocrystallization, inclusion, and liposome formation. There are benefits and negatives associated with each of these technologies that should be considered when selecting the appropriate encapsulation technology for a certain medicament. The method of encapsulation used is not narrowly critical to the invention. Any encapsulation method now known or any equivalent encapsulation method to be known in the future can be used to prepare encapsulated therapeutic substances as disclosed herein.

Cocrystallization is an example of a process that can be utilized to create this invention. Cocrystallization is defined as a process whereby a second ingredient is incorporated into a micro-sized sucrose crystal by the unique phenomenon of spontaneous crystallization (Rizzuto et al., 1984). Various techniques and apparatuses have been developed for carrying out the continuous crystallization of highly water-soluble solutes, such as sugar (U.S. Pat. Nos. 1,724,627, 2,160,533, 2,587,293, 3,194,682, 3,247,021, 3,365,331, 3,424,221, 3,503,803, 3,627,582 and 3,680,621). Further improvements were made to the disclosed apparatuses to decrease the cost and complexity and increase the commercial viability of cocrystallizated products (U.S. Pat. Nos. 3,981,739 and 4,009,045). U.S. Pat. Nos. 4,159,210, 4,338,350, and 4,362,757 disclose applications of cocrystallized products for the food, flavor, and pharmaceutical industries.

Cocrystallization involves modifying sucrose crystals into an irregular, micro-sized agglomerated form that traps flavors or other ingredients (Chen et al., 1988). Spontaneous crystallization occurs when a supersaturated sugar solution is vigorously agitated. Adding flavors or other additives causes the mixture to crystallize and traps the components in the matrix. The methods described in U.S. Pat. No. 4,338,350 were used to create examples of this invention.

Cocrystallization leads to stable products with a very good shelf life. Other benefits include direct tabletting characteristics which provide significant advantages to the candy and pharmaceutical industries (Rizzuto et al., 1984). Cocrystallization also improves the solubility, dispersability, wetability, anticaking, antiseparation, antidusting, homogeneity, flowability of the product. Furthermore, the medicament is evenly dispersed in micro-sized particles entrapped within the flavor/sugar matrix. As the product dissolves in the mouth, the small size of the medicament and its proximity to high sugar and flavor levels prevents the perception of any off tastes. This allows products formulated with the encapsulated medicament to be held in the mouth and absorbed in the oral cavity.

It was mentioned that the bitter taste imparted by many drugs and the psychological barriers associated with holding drugs prevents the general use of orally absorbed medicines. Lozenge formulations overcome these issues because the high sweetness and flavor levels reminds patients of candy. The present invention goes one step further by encapsulating flavor, sweetener, and medicament. This provides patients with an acceptable and even pleasant delivery form, leading to better patient compliance.

### EXAMPLE 1

This example describes the encapsulation of aspirin via cocrystallization. Five hundred grams of granulated sugar and 150 grams of water were heated to 255° F. The molten sugar was transferred to a paddle mixer (Kitchen Aide) and continuously stirred. Fifty grams of granulated USP Aspirin 1020 (Rhodia, Cranbury, N.J.) was gradually added. Approximately one teaspoon (2.76 grams) of peppermint flavoring (Wilton Enterprises, Woodridge, Ill.) was added along with 10 drops of liquid blue food coloring (McCormick & Co., Hunt Valley, Md.). The flavor in combination with the vigorous mechanic agitation provides nucleation for the sucrose/aspirin mixture to crystallize. Within a couple of minutes, the syrup reached the temperature at which transformation and crystallization can begin and the mixture is converted into a powder. A significant amount of latent heat was given off during crystallization, leading to evaporation of the moisture and creation of a dry product. The product is compressible and can be formed into tablets using conventional methods known to those in the pharmaceutical and confectionery field. A 1.1 gram tablet provides 100 mg of aspirin and has a pleasant, sweet, mint character.

### EXAMPLE 2

This example describes the encapsulation of caffeine via cocrystallization. To a mixture of 77 grams of anhydrous caffeine (AMC Chemicals, Avenel, N.J.) and 500 grams of sucrose was added 150 grams of water. The mixture was heated to 255° F. and the syrup was transferred to the mixer and continually stirred. Peppermint oil (5.8 grams, U.S. Ingredients, Aurora, Ill.), and 10 drops of liquid blue food coloring are added. The mixture passes through a sieve mesh #60 and is compressed into 750 mg tablets. Each tablet provides 100 mg of caffeine and has a pleasant, sweet, mint character.

### EXAMPLE 3

This example describes the creation of concentrated caffeine crystals, encapsulated with flavor and color, using cocrystallization. Sugar (200 grams), anhydrous caffeine (73.74 grams), and water (50 grams) are heated until 255° F., following the process detailed in Example One. Peppermint oil (2.76 grams, U.S. Ingredients, Aurora, Ill.) and 10 drops liquid blue food coloring are added. A blend of 75 mg of the encapsulated drug and 675 mg of tabletting sugar (Di-Pac®, Domino Sugar Corp., New York, N.Y.) are compressed into a tablet yielding 20 mg of caffeine.

### EXAMPLE 4

This example describes the encapsulation of estradiol via cocrystallization. Five hundred grams of granulated sugar and 150 grams of water are heated to 255° F., following the process detailed in Example One. Estradiol (0.167 grams) is added to the mixture along with flavor (5.1 grams of peppermint oil, U.S. Ingredients, Aurora, Ill.) and color (10 drops of liquid blue food coloring, McCormick & Co., Hunt Valley, Md.). The mixture passes through a sieve mesh #60 and is compressed into 750 mg tablets. Each tablet provides 0.25 mg of estradiol for estrogen therapy and has a pleasant, sweet, mint character. Estrogens, such as estradiol, have been shown to be ten to twenty times more effective sublingually than orally (Miescher and Gasche, 1942).

### EXAMPLE 5

This example describes the encapsulation of estradiol via extrusion, as described in U.S. Pat. No. 5,786,017. Saccharose (42.6%), maltodextrin (42.6%), estradiol (0.34%), water (3.96%), peppermint oil (10.0%), lecithin (0.5%), and FD&C Blue 1 (0.001%) are blended together. The powder is fed into a twin screw cooker extruder and heated to 150° C. The mixture is extruded, cooled, ground into rods, and screened. The resulting crystals, containing the drug, flavor, and color, are combined with tabletting sugar (Di-Pac®, Domino Sugar Corp., New York, N.Y.). A blend of 75 mg of the encapsulated drug and 675 mg of the tabletting sugar can be compressed into tablets that yield 0.25 mg estradiol.

### EXAMPLE 6

This example describes the encapsulation of ibuprofen through cocrystallization. Five hundred grams of sucrose and 150 grams of water are heated to 255° F. Ibuprofen (126.58 grams) and peppermint oil (6.33 grams) are added while vigorously mixed. The resulting powder passes through a sieve mesh #60 and is compressed into 750 mg tablets. Each tablet provides 150 mg of ibuprofen.

Ibuprofen is a non-steroidal anti-inflammatory drug that reduces the level of prostaglandins in the body. Ibuprofen has been shown to be an effective treatment for fever, cold and flu, arthritis, gout, back aches, dental pain, head ache, migraine, muscle aches and pains, sinusitis.

Ibuprofen has a high lipid/oil partition coefficient, allowing it to be readily absorbed through the lipid membranes. Lower levels are required due to the rapid absorption through the oral mucosa. Encapsulation of the ibuprofen with flavor and sweetener masks the bitterness of the drug, allowing it to be palatable to the consumer.

### EXAMPLE 7

This example describes the encapsulation of progesterone through cocrystallization. Five hundred grams of sucrose and 150 grams of water are heated to 255° F. Progesterone (126.58 grams) and peppermint oil (6.33 grams) are added while vigorously mixed. The resulting powder passes through a sieve mesh #60 and is compressed into 750 mg tablets. Each tablet provides 150 mg of progesterone.

Postmenopausal estrogen replacement therapy is often prescribed, but current treatments suffer from poor patient compliance as many women object to some of the side-effects and the inconvenience of the pharmaceutical forms of the medication. Sublingual applications of progesterone have been shown to be 50% more effective than oral applications (Joel, 1942). By combining the hormone with flavor and sweetener, this invention provides a pleasant tasting and convenient delivery form for those on hormone replacement therapy.

### EXAMPLE 8

This example describes the encapsulation of warfarin, an anticoagulant used for the prevention and treatment of blood clots and heart attacks, using cocrystallization. The procedure of Example One is repeated using 17.42 grams of sodium warfarin and 5.23 grams of peppermint oil (U.S. Ingredients, Aurora, Ill.). The resulting 750 mg tablet contains 25 mg of warfarin.

### EXAMPLE 9

This example describes the encapsulation of nicotine using cocrystallization. The procedure of Example Three is repeated using 5.54 grams of nicotine and 2.08 grams of peppermint oil (U.S. Ingredients, Aurora, Ill.). A blend of 75 mg of the encapsulated drug and 675 mg of tabletting sugar (Di-Pac®, Domino Sugar Corp., New York, N.Y.) are compressed into a tablet yielding 2 mg of nicotine. Nicotine passes through the oral mucosa with considerable ease and the tablet can be used to help patients stop smoking.

### EXAMPLE 10

This example describes the encapsulation of diphenhydramine, an antihistamine and sedative, via spray drying. One hundred grams of diphenhydramine hydrochloride is combined with 500 grams of water and 200 grams of an enzymatically converted starch derivative, an example of which is described by U.S. Pat. No. 6,086,917. The mixture is heated to 60° C. until starch dissolution is complete and then lowered to 40° C. Peppermint oil (75 grams) is added and emulsified at high speed for approximately three minutes. The emulsion is then spray dried into a powder using standard techniques known to those who practice the art. The resulting powder is combined with tableting sugar (5%:95% w/w) and compressed into tablets with a lubricating agent, such as magnesium stearate. The resulting 750 mg tablet contains 10 mg of diphenhydramine.

### EXAMPLE 11

This example describes the encapsulation of acetaminophen, a common pain reliever and fever reducer, using coated rotogranules. The rotogranulating and coating process is described in U.S. Pat. No. 5,460,825. Rotogranules are created by spraying water, at a rotor speed of 400-500 RPM, onto a mixture of 200 grams of acetaminophen, 7.5 grams of artificial grape oil, 18.75 grams of hydroxypropylmethyl cellulose, and 111.24 grams of sucrose. The granules are dried to 30-35° C., at a rotor speed of 250 RPM. The granules are coated using 37.5 grams of a solution by weight of a 70:30 blend of cellulose acetate to hydroxypropyl cellulose. The coated granules are combined with 375 grams of compressible sugar and formed into 750 mg tablets yielding 200 mg of acetaminophen.

### EXAMPLE 12

This example describes the encapsulation of phenmetrazine, an appetite suppressant, via cocrystallization. Phenmetrazine (9.70 grams) is added to 500 grams of sorbitol (70% solution, Archer Daniels Midland, Decatur, Ill.). The mixture is heated to 255° F. and the syrup is transferred to a mixer and continually stirred. Peppermint oil (3.63 grams, U.S. Ingredients, Aurora, Ill.), and 10 drops of liquid blue food coloring are added. The mixture passes through a sieve mesh #60 and is compressed into 750 mg tablets. Each tablet provides 20 mg of phenmetrazine.

### EXAMPLE 13

This example describes the encapsulation of benzocaine using cocrystallization. Benzocaine is a reducing aid used by those controlling their diet. The procedure of Example Three is repeated using 17.58 grams of benzocaine and 2.20 grams of peppermint oil (U.S. Ingredients, Aurora, Ill.). A blend of 75 mg of the encapsulated drug and 675 mg of sorbitol (Archer Daniels Midland, Decatur, Ill.) are compressed into a tablet yielding 6 mg of benzocaine.

### EXAMPLE 14

This example describes the encapsulation of phenylephrine hydrochloride via extrusion. Phenylephrine hydrochloride stimulates the sympathetic nervous system, lowers blood pressure, and constricts blood vessels. The encapsulation process detailed in Example Five is repeated using 36.0% saccharose, 36.0% maltodextrin, 0.13% of phenylephrine hydrochloride and 4.2% of water. A blend of 75 mg of the encapsulated drug and 675 mg of the tabletting sugar (Di-Pac®) can be compressed into tablets that yield 10 mg phenylephrine hydrochloride.

### EXAMPLE 15

This example describes the encapsulation of amobarbital, a barbiturate, through the process of cocrystallization. The process described in Example Two is repeated using 77 grams of amobarbital. Each 750 mg tablet provides 100 mg of amobarbital.

### EXAMPLE 16

This example describes the encapsulation of phenylpropanolamine hydrochloride (PPA) using cocrystallization. PPA stimulates the sympathetic nervous system and a bronchodilator that is used as a nasal decongestant and dieting aid. The procedure of Example Eight is repeated using 17.42 grams of PPA and the resulting 750 mg tablet contains 25 mg of PPA.

### EXAMPLE 17

This example describes the encapsulation of dimenhydrinate using cocrystallization. Dimenhydrinate is an antihistamine used to prevent nausea and vomiting associated with “morning” sickness in pregnant women and motion sickness. The procedure of Example Eight is repeated using 17.42 grams of dimenhydrinate and the resulting 750 mg tablet contains 25 mg of dimenhydrinate.

### EXAMPLE 18

This example describes the encapsulation of pseudoephedrine hydrochloride using cocrystallization. Pseudoephedrine hydrochloride is a vasoconstrictor and bronchodilator and is used as a nasal decongestant and for the treatment of allergies. The procedure of Example Eight is repeated using 17.42 grams of pseudoephedrine. The resulting 750 mg tablet contains 25 mg of pseudoephedrine.

### EXAMPLE 19

This example describes the encapsulation of meclizine using cocrystallization. Meclizine is antihistamine and is used for the treatment of motion sickness. The procedure of Example Eight is repeated using 17.42 grams of meclizine. The resulting 750 mg tablet contains 25 mg of meclizine.

### EXAMPLE 20

This example describes the encapsulation of phenobarbital sodium using cocrystallization. Phenobarbital sodium is a long acting sedative, hypnotic, and anticonvulsant. The procedure of Example Eight is repeated using 17.42 grams of phenobarbital sodium. The resulting 750 mg tablet contains 25 mg of phenobarbital sodium.

### EXAMPLE 21

This example describes the encapsulation of testosterone using cocrystallization. Testosterone is a steroid and an anti-estrogenic hormone. The procedure of Example Eight is repeated using 17.42 grams of testosterone. The resulting 750 mg tablet contains 25 mg of testosterone.

### EXAMPLE 22

This example describes the encapsulation of prednisolone using cocrystallization. Prednisolone is an immuno-suppressant and used for the treatment for severe inflammation. The procedure of Example Eight is repeated using 17.42 grams of prednisolone. The resulting 750 mg tablet contains 25 mg of prednisolone.

### EXAMPLE 23

This example describes the encapsulation of prednisone using cocrystallization. Prednisone is an immuno-suppressant and used for the treatment for severe inflammation, acute attacks of multiple sclerosis, arthritis, and irritable bowel syndrome. The procedure of Example Eight is repeated using 17.42 grams of prednisone. The resulting 750 mg tablet contains 25 mg of prednisone.

### EXAMPLE 24

This example describes the encapsulation of diazepam, a tranquilizer and skeletal muscle relaxant, using cocrystallization. The encapsulation procedure of Example Nine is repeated using 5.54 grams of diazepam, yielding a tablet with 2 mg of diazepam.

### EXAMPLE 25

This example describes the encapsulation of dyclonine hydrochloride, an anesthetic that relieves minor sore throat and mouth pain, using cocrystallization. The encapsulation procedure of Example Nine is repeated using 5.54 grams of dyclonine hydrochloride, which yields a tablet with 2 mg of dyclonine hydrochloride.

### EXAMPLE 26

This example describes the encapsulation of chlorpheniramine using coacervation. Chlorpheniramine is an antihistamine used as a decongestant and treatment of allergy symptoms. A solution of 80 ml of ethanol and 5.0 grams of Castor Wax A (mp 85° C.) is boiled to 78° C. as detailed in U.S. Pat. No. 4,102,806. The mixture is transferred to a propeller mixer and 0.77 grams of chlorpheniramine and 1.44 grams of peppermint oil are added. The encapsulated drug and flavor are centrifuged to remove the ethanol and dried under reduced pressure and at 40° C. for three hours. The resulting powder is screened through a 60 mesh sieve. The resulting material is combined with tableting sugar (5%:95% w/w ratio) and compressed into 750 mg tablets, such that each tablet contains 4 mg of chlorpheniramine.

### EXAMPLE 27

This example describes the encapsulation of cyclizine hydrochloride via cocrystallization. Cyclizine hydrochloride is an antihistamine used in the prevention and treatment of nausea, vomiting, and dizziness associated with motion sickness. 500 grams of sucrose and 150 grams of water are heated to 255° F. The syrup is transferred to the mixer and continually stirred. Peppermint oil (5.41 grams, U.S. Ingredients, Aurora, Ill.), cyclizine hydrochloride (36.10 grams), and 20 drops of liquid blue food coloring are added. The mixture passes through a sieve mesh #60 and is compressed into 750 mg tablets. Each tablet provides 50 mg of cyclizine hydrochloride.

### EXAMPLE 28

This example describes the encapsulation of medicines for the treatment of a hangover, including a combination of pain reliever and stimulants. The medicines are encapsulated via cocrystallization. A mixture of 300 grams of sucrose and 100 grams of water is heated to 255° F. and transferred to a mixer. The following components are added slowly while vigorously mixing: 122.30 gram of aspirin (Rhodia, Cranbury, N.J.), 30.58 grams of anhydrous caffeine (AMC Chemicals, Avenel, N.J.), 5.73 grams of peppermint oil (U.S. Ingredients, Aurora, Ill.), and 20 drops of blue food coloring (McCormick & Co., Hunt Valley, Md.). The resulting powder is compressed into tablets, each providing 200 mg aspirin and 50 mg of caffeine, for fast acting relief of a headache or hangover.

While the invention has been described with reference to specific examples and processes, these were for purposes of illustration only and should not be construed to limit the scope of the present invention.

What is claimed is:

1. A dry particulate drug delivery system for dissolution of pharmaceutical compounds in the mouth, comprising:
a therapeutically-effective amount of at least one drug, and an encapsulant, wherein said at least one drug is encapsulated by said encapsulant.

2. The dry particulate drug delivery system of claim 1, further comprising at least one volatile flavorant.

3. The dry particulate drug delivery system of claim 1, further comprising at least one sweetener.

4. The dry particulate drug delivery system of claim 3, further comprising at least one volatile flavorant.

5. The dry particulate drug delivery system of claim 1, wherein said encapsulant is a sucrose-based matrix.

6. The dry particulate drug delivery system of claim 1, wherein said therapeutically effective amount of said at least one drug is dependent on the desired dosage, method of encapsulation, absorption characteristics, and desired tablet size.

7. The dry particulate drug delivery system of claim 1, wherein said drug is a therapeutic agent selected from the group consisting essentially of anesthetics, decongestants, antihistamines, antitussives, antibotics, analgesics, alkaloids, amphetamines, hormones, barbiturates, analeptics and mixtures thereof.

8. The dry particulate drug delivery system of claim 7, wherein said alkaloids are selected from the group consisting essentially of morphine, codeine, caffeine, and cocaine.

9. The dry particulate drug delivery system of claim 7, wherein said hormones are selected from the group consisting essentially of adrenaline and epinephrine.

10. The dry particulate drug delivery system of claim 3, wherein said at least one sweetener is selected from the group consisting essentially of sucrose, fructose, dextrose, lactose, mannitol, sorbitol, maltose, xylitol, saccharin and saccharin sodium, cyclamates, neotame, aspartame, sucralose, acesulfame potassium and mixtures thereof.

11. The dry particulate drug delivery system of claim 1, further comprising at least one of natural and artificial sweeteners, colors, preservatives, fillers, binders, lubricants, suspending agents, acids, buffers, and stabilizing agents.

12. The dry particulate drug delivery system of claim 1, wherein said system is a compressed tablet.

13. The dry particulate drug delivery system of claim 1, wherein said system is a chewing gum.

14. The dry particulate drug delivery system of claim 1, wherein said drug is encapsulated by a method selected from the group consisting essentially of spray drying, air suspension coating, extrusion, spray cooling and chilling, emulsion, microemulsion, adsorption, centrifugal extrusion, rotational suspension separation, rotogranulation, coacervation, continuous crystallization, cocrystallization, inclusion, and liposome formation.

15. The dry particulate drug delivery system of claim 14, wherein aspirin is encapsulated by said cocrystallization method.

16. The dry particulate drug delivery system of claim 14, wherein caffeine is encapsulated by said cocrystallization method.

17. The dry particulate drug delivery system of claim 14, wherein estradiol is encapsulated by said cocrystallization method.

18. The dry particulate drug delivery system of claim 14, wherein estradiol is encapsulated by said extrusion method.

19. The dry particulate drug delivery system of claim 14, wherein ibuprofen is encapsulated by said cocrystallization method.

20. The dry particulate drug delivery system of claim 14, wherein progesterone is encapsulated by said cocrystallization method.

21. The dry particulate drug delivery system of claim 14, wherein warfarin is encapsulated by said cocrystallization method.

22. The dry particulate drug delivery system of claim 14, wherein nicotine is encapsulated by said cocrystallization method.

23. The dry particulate drug delivery system of claim 14, wherein diphenhydramine is encapsulated by said spray drying method.

24. The dry particulate drug delivery system of claim 14, wherein acetaminophen is encapsulated by said rotogranulating method.

25. The dry particulate drug delivery system of claim 14, wherein phenmetrazine is encapsulated by said cocrystallization method.

26. The dry particulate drug delivery system of claim 14, wherein benzocaine is encapsulated by said cocrystallization method.

27. The dry particulate drug delivery system of claim 14, wherein phenylephrine hydrochloride is encapsulated by said cocrystallization method.

28. The dry particulate drug delivery system of claim 14, wherein amobarbital is encapsulated by said cocrystallization method.

29. The dry particulate drug delivery system of claim 14, wherein phenylpropanolamine hydrochloride is encapsulated by said cocrystallization method.

30. The dry particulate drug delivery system of claim 14, wherein dimenhydrinate is encapsulated by said cocrystallization method.

31. The dry particulate drug delivery system of claim 14, wherein pesudoephedrine hydrochloride is encapsulated by said cocrystallization method.

32. The dry particulate drug delivery system of claim 14, wherein meclizine is encapsulated by said cocrystallization method.

33. The dry particulate drug delivery system of claim 14, wherein Phenobarbital sodium is encapsulated by said cocrystallization method.

34. The dry particulate drug delivery system of claim 14, wherein testosterone is encapsulated by said cocrystallization method.

35. The dry particulate drug delivery system of claim 14, wherein prednisolone is encapsulated by said cocrystallization method.

36. The dry particulate drug delivery system of claim 14, wherein diazepam is encapsulated by said cocrystallization method.

37. The dry particulate drug delivery system of claim 14, wherein dyclonine hydrochloride is encapsulated by said cocrystallization method.

38. The dry particulate drug delivery system of claim 14, wherein chlorpheniramine is encapsulated by said coacervation method.

39. The dry particulate drug delivery system of claim 14, wherein cyclizine hydrochloride is encapsulated by said cocrystallization method.

40. The method of dissolving a pharmaceutical compound in the mouth of a user, comprising the steps of:
encapsulating a therapeutically-effective amount of at least one drug within an encapsulant and forming an encapsulated drug,
inserting said encapsulated drug into the mouth of the user,
subjecting said encapsulated drug to fluids secreted within the mouth of said user,
dissolving said encapsulant and releasing said drug in said mouth,
dissolving said drug within said mouth of said user.
